SEQUENCE LISTING (7 sequences)

SEQ ID NO: 1 - protein, 118 aa, synthetic construct
MDYKDDDDKMVSRDQAHLGPKYVGLWDFKRSTDEELSFRAGDVFHVARKEEQWWWATLLDEAGGAVAQGYVPHNYLAERETVESEPAGHAGSAALQDLAASRGPAAPERGGLPQPARA

SEQ ID NO: 2 - protein, 100 aa, synthetic construct
MDYKDDDDKMVSRDQAHLGPKYVGLWDFKSRTDEELSFRAGDVFHVARKEEQWWWATLLDEAGGAVAQGYVPHNYLAERETVESEPAGHAGCAALQDLAA

SEQ ID NO: 3 - protein, 84 aa, synthetic construct
MDYKDDDDKMVSRDQAHLGPKYVGLWDFKSRTDEELSFRAGDVFHVARKEEQWWWATLLDEAGGAVAQGYVPHNYLAERETVES

SEQ ID NO: 4 - protein, 83 aa, synthetic construct
MVSRDQAHLGPKYVGLWDFKSRTDEELSFRAGDVFHVARKEEQWWWATLLDEAGGAVAQGYVPHNYLAERETVESEPAGHAGS

SEQ ID NO: 5 - protein, 91 aa, synthetic construct
MVSRDQAHLGPKYVGLWDFKSRTDEELSFRAGDVFHVARKEEQWWWATLLDEAGGAVAQGYVPHNYLAERETVESEPAGHAGCAALQDLAA

SEQ ID NO: 6 - protein, 91 aa, synthetic construct
MVSRDQAHLGPKYVGLWDFKSRTDEELSFRAGDVFHVARKEEQWWWATLLDEAGGAVAQGYVPHNYLAERETVESEPAGHAGSAALQDLAA

SEQ ID NO: 7 - protein, 100 aa, synthetic construct
RRRRRRRRRMVSRDQAHLGPKYVGLWDFKSRTDEELSFRAGDVFHVARKEEQWWWATLLDEAGGAVAQGYVPHNYLAERETVESEPAGHAGSAALQDLAA

(19) United States
(12) Patent Application Publication
(10) Pub. No.: US 2026/0234204 A1
(43) Pub. Date: Aug. 13, 2026

(54) BRK PEPTIDES AND METHODS OF USE

(71) Applicant: Concarlo Therapeutics, Inc., Brooklyn, NY (US)

(72) Inventor: Grace Chen, Brooklyn, NY (US)

(21) Appl. No.: 19/471,680

(22) PCT Filed: Apr. 12, 2024
(86) PCT No.: PCT/US2024/024494

§ 371 Date: Oct. 1, 2025

Related U.S. Application Data
(60) Provisional application No. 63/459,558, filed on Apr. 14, 2023.

(51) Int. Cl. C07K 14/47; A61K 9/51; A61K 38/00; A61K 45/06; A61P 35/00

(52) U.S. Cl. CPC C07K 14/4703; A61K 9/5123; A61K 45/06; A61P 35/00; A61K 38/00

(57) ABSTRACT

The invention relates to truncated isolated BRK peptides and functional peptides thereof that inhibit the phosphorylation of p27Kip1 and the resulting kinase activity of CDK2 and CDK4; and to pharmaceutical compositions thereof. The invention further relates to the use of the isolated peptides in methods of treating cancer in subject in need thereof. The methods of treating cancer include methods of treating pancreatic cancer.

### CROSS REFERENCE TO RELATED APPLICATIONS

This application claims the benefit under 35 U.S.C. § 119(e) of U.S. Provisional Patent Application No. 63/459,558, filed Apr. 14, 2023. The content of this application is incorporated herein by reference in its entirety for all purposes.

### INCORPORATION OF SEQUENCE LISTING

The material in the accompanying sequence listing is hereby incorporated by reference into this application. The accompanying sequence listing xml file, name 395334_000031SequenceListing.xml, was created on Mar. 26, 2024, and is 8,192 bytes in size.

### BACKGROUND OF THE INVENTION

### Field of the Invention

The present invention relates generally to peptides and more specifically to truncated peptides and uses thereof to inhibit cancer cell proliferation.

### Background Information

p27 interacts specifically with CDK4/6 and CDK2 and is responsible for turning these kinases OFF and ON. This transition from OFF to ON is mediated by a specific modification to p27 itself, by the tyrosine kinase BRK (Breast tumor Related Kinase). A naturally occurring alternatively spliced form of BRK (ALT), which removes the BRK kinase domain, permits binding to p27, blocking BRK's association, and preventing BRK's phosphorylation of p27. This locks CDK4/6 and CDK2 into the OFF conformation and simultaneously inhibits the activity of both kinases.

CDK4/6 inhibitors (CDK4/6i) are now the standard of care for ER+, metastatic breast cancer. However, acquired resistance to these drugs remains an issue. Pancreatic ductal adenocarcinoma (PDAC) is characterized by aberrant activation of the Cyclin D1-CDK4/6 (CDK4/6) signaling pathway, due to mutation of RAS and inactivation of the CDKN2A locus encoding the CDK4/6 inhibitor p16INK4A. A priori, this tumor type might have seemed like a good candidate for the use of CDK4/6i. However, CDK4/6i's show only modest activity in this tumor type, suggesting a resistance to this form of therapy.

Here a novel strategy to inhibit the CDK4/6 pathway using a therapeutic liposomal:peptide formulation to inhibit the tyrosine phosphorylation of p27KIP1, which in turn inhibits both CDK4/6 and CDK2 is described.

### SUMMARY OF THE INVENTION

The present invention is based on the seminal discovery that truncated ALT peptides, rather than the full-length peptide, inhibits pancreatic cancer cell proliferation and survival. Such peptides are incorporated into pharmaceutical compositions and used for the treatment of pancreatic cancer in a subject in need thereof.

In one embodiment, the invention provides a method of treating pancreatic cancer in a subject in need thereof including administering to the subject a therapeutically effective amount of a pharmaceutical composition including an isolated peptide having the amino acid sequence of SEQ ID NO:6, thereby treating pancreatic cancer in the subject.

In one aspect, the peptide inhibits cancer cell proliferation and/or decreases cancer cell viability. In another aspect, the peptide inhibits tumor growth. In one aspect, the peptide increases cancer cell death. In another aspect, the peptide increases tumor necrosis. In one aspect, the peptide increases cancer cells reactive oxygen species (ROS) level. In one aspect, the peptide inhibits phosphorylation of p27. In another aspect, the peptide inhibits CDK2 and CDK4.

In various aspects, the pancreatic cancer is pancreatic ductal adenocarcinoma (PDAC). In some aspects, the PDAC is cyclin-dependent kinase 4/6 (CDK4/6) inhibitor resistant.

In one aspect, the pharmaceutical composition further includes a delivery vehicle. In some aspects, the delivery vehicle is selected from the group consisting of a nanoparticle, a liposome, a dendrimer, a micelle, a nanoemulsion, a nanosuspension, a niosome, a nanocapsule, a magnetic nanoparticle, a lipoprotein-based carrier, and a lipoplex nanoparticle. In various aspects, the lipoplex nanoparticle includes 1,2-di-O-octdecenyl-3-trimethyl ammonium propane (DOTMA), cholesterol, DOPE, TPGS, or a combination thereof. In other aspects, the lipoplex nanoparticle includes DOTMA and cholesterol. In other aspects, the lipoplex nanoparticle includes DOTMA, cholesterol and TPGS.

In one aspect, the method further includes administering to the subject an anti-cancer treatment. In some aspects, the anti-cancer treatment is selected from the group consisting of chemotherapy, radiation treatment, immunotherapy, resection of a tumor, and any combination thereof. In other aspects, the anti-cancer treatment is administered prior to, simultaneously with, or after administration of the pharmaceutical composition. In some aspects, the anti-cancer treatment is an anti-cancer agent selected from the group consisting of palbociclib, ribociclib, abemaciclib, osirmetinib, gefitinib, lapatinib, pantitumumab, vandetanib, necitumumab, vemurafenib, sorafenib tosylate, PLX-4720, dabrafenib, paclitaxel, cisplatin, docetaxol, carboplatin, vincristine, vinblastine, methotrexate, cyclophosphamide, CPT-11, 5-fluorouracil, gemcitabine, estramustine, carmustine, adriamycin, etoposide, arsenic trioxide, irinotecan, vemurafenib, erlotinib, cetuximab, letrozole, fulvestrant, and epolhilone derivatives.

In another embodiment, the invention provides a method of treating pancreatic cancer in a subject in need thereof including administering to the subject a therapeutically effective amount of a pharmaceutical composition including a cyclin-dependent kinase 4/6 (CDK4/6) and CDK2-specific inhibitor, thereby treating pancreatic cancer in the subject.

In one aspect, the CDK4/6 and CDK2-specific inhibitor is an isolated peptide having the amino acid sequence of SEQ ID NO:6.

### BRIEF DESCRIPTION OF THE DRAWINGS

FIGS. 1A-1D. FIGS. 1A and 1B are respectively a graph and an image showing that PDAC cells are resistant to palbociclib-treatment. Cells were treated with increasing concentrations of palbociclib (PD) for 24 h before analysis for viability (Cell Titer Glo, Promega). N=4 *p<0.05. Control=untreated cells. Viability plotted relative to growth in the presence of control. FIGS. 1C and 1D show PL5 cells treated with increasing concentrations of IpY.20 and E′NP before analysis of Cell Titer Glo analysis.

FIGS. 2A-2F. FIGS. 2A and 2B are Western Blots, illustrating results from assays performed with treatment naive cells to analyze the expression levels of p27. Cells were harvested with RIPA buffer supplemented with protease inhibitors. The MCF7-ALT Dox inducible cell line was used as a control. FIG. 2C shows that IpY.20 reduces viability in CDK4i resistant pancreatic cell lines. Cells were treated with increasing concentrations of IpY.20 for 24h. Viability was measured using the Cell Titer Glo assay (Promega). Viability was calculated relative to untreated control cells. N=4 *p<0.05. The MCF7-ALT inducible cells showed to reduced viability after 48 h of Dox induction. PL5, Panc1, L3.6 and BXPC3 exhibited super sensitivity (SS) to IpY.20 treatment. FIGS. 2D, 2E and 2F are graphs illustrating that IpY.20 induces ROS. Cells were treated with IpY.20 for 24 h. ROS levels were measured using ROS-Glo assay (Promega). % ROS was calculated relative to untreated control cells. N=4 *p<0.05. The MCF7-ALT inducible cell line induced ROS.

FIGS. 3A-3F are images, graphs, and a diagram showing that IpY.20 induces necroptosis, which is rescued by NEC-1 and NAC treatment.

FIGS. 4A-4G are images and graphs showing that IpY20 induces apoptosis.

FIGS. 5A-5C are graphs showing that cell lines show different uptake kinetics which correlated with IpY sensitivity. In FIG. 5A, cells were treated with different concentrations of IpY.1 for 24 h. In FIG. 5B, cells were treated with 2.5 ng/μl of IpY.1 for 2, 4, 6 and 8 h. In both assays, L3.6 (a super sensitive cell line) showed higher uptake capacity when compared with MiaPaCa. In FIG. 5C, MiaPaCa cells were treated with 7.5 ng/μl of IpY.1 for 2 hours and L3.6 with 2.5 ng/μl of IpY.1 for the same period of time to achieve the same intracellular concentration of IpY1 at time 0. After removing IpY.1 from the media after 1 hr. (time 0), samples were collected every 2 h for further analysis. Peptide was lost from MiaPaCa and L3.6 with similar kinetics.

FIG. 6 is a schematic diagram summarizing the findings of the invention.

FIGS. 7A-7C are schematic diagrams illustrating the structure of the complex. In FIG. 7A the Complex is closed and off, and the CDK active site is inaccessible. In FIG. 7B, the BRK is bound to phosphate and the Complex is on, and the CDK active site is accessible. In FIG. 7C, the small protein ALT prevents BRK binding and keeps the Complex closed and off.

FIG. 8 is a graph illustrating that IpY.1 blocks tumor cell proliferation. MCF7 cells were treated with ALT peptide (second-fourth from left), empty NP (middle) or the assembled IpY.1 (right) before analysis for proliferation.

FIG. 9 is a set of graphs illustrating tumor volumes (top) and overall survival (bottom) compared between no treatment, palbociclib, IpY, and IpY plus Palbociclib. IpY.1 causes tumor regression and increases overall survival in palbociclib-resistant tumors: MCF7 xenografts were rendered palbociclib-resistant by treatment with 0.75 mg/kg palbociclib for extended periods of time. When tumors≥700 mm³ in the presence of palbociclib, mice were randomly assigned to three different groups: (1) continued palbociclib (75 mg/kg, oral gavage, daily) (second from left); (2) IpY.1 (0.75 mg/kg, i.v., 3×/week) (third from left); (3) palbociclib+IpY.1 (right). Tumor volumes were measure by calibers. Study endpoint: Tumor volumes>1500 mm³ or death.

### DETAILED DESCRIPTION OF THE INVENTION

The present invention is based on the seminal discovery that truncated ALT peptides, rather than the full-length peptide, specifically inhibit pancreatic cancer cell proliferation and survival. Such peptides are incorporated into pharmaceutical compositions and used for the treatment of pancreatic cancer in a subject in need thereof.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” include one or more methods, and/or steps of the type described herein which will become apparent to those persons skilled in the art upon reading this disclosure and so forth.

As used herein, the term “and/or” includes any and all combinations of one or more of the associated listed items.

As used herein, the term “about” in association with a numerical value is meant to include any additional numerical value reasonably close to the numerical value indicated. For example, and based on the context, the value varies up or down by 5-10%. For example, for a value of about 100, means 90 to 110 (or any value between 90 and 110).

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In one embodiment, the invention provides a method of treating pancreatic cancer in a subject in need thereof including administering to the subject a therapeutically effective amount of a pharmaceutical composition including an isolated peptide having the amino acid sequence of SEQ ID NO:6, thereby treating pancreatic cancer in the subject.

The term “subject” as used herein refers to any individual or patient to which the subject methods are performed. Generally, the subject is human, although as will be appreciated by those in the art, the subject may be a non-human animal. Thus, other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

The term “treatment” is used interchangeably herein with the term “therapeutic method” or “therapy” and refers to 1) therapeutic treatments or measures that cure, slow down, lessen symptoms of, and/or halt progression of a diagnosed pathologic conditions or disorder, and/or 2) prophylactic/preventative measures. Those in need of treatment may include individuals already having a particular medical disorder as well as those who may ultimately acquire the disorder (i.e., those needing preventive measures).

The terms “therapeutically effective amount”, “effective dose,” “therapeutically effective dose”, “effective amount,” or the like refer to that amount of the subject compound that will elicit the biological or medical response of a tissue, system, animal or human that is being sought by the researcher, veterinarian, medical doctor or other clinician. Generally, the response is either amelioration of symptoms in a patient or a desired biological outcome (e.g., treatment of the cancer). The “effective amount” of an active agent is an amount that is non-toxic to a subject or a majority or normal cells but is an amount of the active agent that is sufficient to provide a desired effect (e.g., treatment of cancer). This amount may vary from subject to subject, depending on the species, age, and physical condition of the subject, the severity of the disease that is being treated, the particular conjugate, or more specifically, the particular active agent used, its mode of administration, and the like. Therefore, it is difficult to generalize an exact “effective amount,” yet a suitable effective amount may be determined by one of ordinary skill in the art. The effective amount can be determined as described herein.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization. The phrases “parenteral administration” and “administered parenterally” as used herein means modes of administration other than enteral and topical administration.

The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectables, implantable sustained release formulations, lipid complexes, etc.

As used herein, “pharmaceutical composition” refers to a formulation comprising an active ingredient, and optionally a pharmaceutically acceptable carrier, diluent or excipient. The term “active ingredient” can interchangeably refer to an “effective ingredient” and is meant to refer to any agent that is capable of inducing a sought-after effect upon administration. In one embodiment, the active ingredient includes a biologically active molecule. As used herein, the phrase “biologically active molecule” refers to a molecule that has a biological effect in a cell. In certain embodiments the active molecule may be an inorganic molecule, an organic molecule, a small organic molecule, a drug compound, a peptide, a polypeptide, such as an enzyme or transcription factor, an antibody, an antibody fragment, a peptidomimetic, a lipid, a nucleic acid such as a DNA or RNA molecule, a ribozyme, hairpin RNA, siRNA (small interfering RNAs) of varying chemistries, miRNA, siRNA-protein conjugate, an siRNA-peptide conjugate, and siRNA-antibody conjugate, an antagomir, a PNA (peptide nucleic acid), an LNA (locked nucleic acids), or a morpholino. In certain illustrative embodiments, the active agent is a polypeptide or peptide, such as an isolated peptide having the amino acid sequence of SEQ ID NO:6.

By “pharmaceutically acceptable” it is meant the carrier, diluent or excipient must be compatible with the other ingredients of the formulation and not deleterious to the recipient thereof, nor to the activity of the active ingredient of the formulation. Pharmaceutically acceptable carriers, excipients or stabilizers are well known in the art, for example Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG). Examples of carrier include, but are not limited to, liposome, nanoparticles, ointment, micelles, microsphere, microparticle, cream, emulsion, and gel. Examples of excipient include, but are not limited to, anti-adherents such as magnesium stearate, binders such as saccharides and their derivatives (sucrose, lactose, starches, cellulose, sugar alcohols and the like) protein like gelatin and synthetic polymers, lubricants such as talc and silica, and preservatives such as antioxidants, vitamin A, vitamin E, vitamin C, retinyl palmitate, selenium, cysteine, methionine, citric acid, sodium sulfate and parabens. Examples of diluent include, but are not limited to, water, alcohol, saline solution, glycol, mineral oil and dimethyl sulfoxide (DMSO).

The term “pharmaceutically acceptable” refers to the fact that the carrier, diluent or excipient must be compatible with the other ingredients of the formulation and not deleterious to the recipient thereof. For example, the carrier, diluent, or excipient or composition thereof may be administered to a subject along with a conjugate of the invention without causing any undesirable biological effects or interacting in an undesirable manner with any of the other components of the pharmaceutical composition in which it is contained.

The pharmaceutical compositions can be administered either alone or in combination with other therapeutic agents, may conveniently be presented in unit dose form and may be prepared by any of the methods well known in the art of pharmacy. All methods include bringing the isolated peptide into association with the carrier, which constitutes one or more accessory ingredients. In general, the pharmaceutical compositions are prepared by uniformly and intimately bringing the active ingredient into association with a liquid carrier. In a pharmaceutical composition, the isolated peptide is included in an amount sufficient to produce the desired effect upon the process or condition of disease.

Techniques for formulation and administration are generally known in the art. Suitable routes may, for example, parenteral delivery, including intramuscular, subcutaneous, intramedullary, intrathecal, intraventricular, intravenous, or intraperitoneal. For injection, the pharmaceutical compositions of the invention may be formulated in aqueous solutions, preferably in physiologically compatible buffers such as Hanks' solution, Ringer's solution, or physiologically buffered saline.

The methods described herein include the administration of an isolated peptide having the amino acid sequence of SEQ ID NO:6.

The terms “peptide”, “polypeptide” and “protein” are used interchangeably herein and refer to any chain of at least two amino acids, linked by a covalent chemical bound. As used herein polypeptide can refer to the complete amino acid sequence coding for an entire protein or to a portion thereof. A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

The isolated peptides described herein have a sequence having at least 80% sequence identity with the amino acid sequence of SEQ ID NO:6. The terms “sequence identity” or “percent identity” are used interchangeably herein. To determine the percent identity of two polypeptide molecules or two polynucleotide sequences, the sequences are aligned for optimal comparison purposes (e.g., gaps can be introduced in the sequence of a first polypeptide or polynucleotide for optimal alignment with a second polypeptide or polynucleotide sequence). The amino acids or nucleotides at corresponding amino acid or nucleotide positions are then compared. When a position in the first sequence is occupied by the same amino acid or nucleotide as the corresponding position in the second sequence, then the molecules are identical at that position. The percent identity between the two sequences is a function of the number of identical positions shared by the sequences (i.e., % identity-number of identical positions/total number of positions (i.e., overlapping positions)×100). In some embodiments the length of a reference sequence (e.g., SEQ ID NO:6) aligned for comparison purposes is at least 80% of the length of the comparison sequence, and in some embodiments is at least 90% or 100%. In an embodiment, the two sequences are the same length.

Ranges of desired degrees of sequence identity are approximately 80% to 100% and integer values in between. Percent identities between a disclosed sequence and a claimed sequence can be at least 80%, at least 85%, at least 90%, at least 95%, at least 96%, at least 97%, at least 98%, at least 99%, at least 99.5%, or at least 99.9%. In general, an exact match indicates 100% identity over the length of the reference sequence (e.g., SEQ ID NO:6). Polypeptides and polynucleotides that are about 85, 86, 87, 88, 89, 90, 91, 92, 93, 94, 95, 96, 97, 98, 99 99.5% or more identical to polypeptides and polynucleotides described herein are embodied within the disclosure. For example, a polypeptide can have 80%, 85%, 90%, 91%, 92%, 93%, 94%, 95%, 96%, 97%, 98%, 99% or more identity to SEQ ID NO:6.

Variants of the disclosed sequences also include peptides, or full-length protein, that contain substitutions, deletions, or insertions into the protein backbone, that would still leave at least about 70% homology to the original protein over the corresponding portion. A yet greater degree of departure from homology is allowed if like-amino acids, i.e., conservative amino acid substitutions, do not count as a change in the sequence. Examples of conservative substitutions involve amino acids that have the same or similar properties. Illustrative amino acid conservative substitutions include the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine to leucine.

In one aspect, the peptide inhibits cancer cell proliferation and/or decreases cancer cell viability. In another aspect, the peptide inhibits tumor growth. In one aspect, the peptide increases cancer cell death. In another aspect, the peptide increases tumor necrosis.

In one aspect, the peptide inhibits phosphorylation of p27. In another aspect, the peptide inhibits CDK2 and CDK4.

The peptides of the present invention are truncated ALT peptides, which are p27 mimetics that inhibit the phosphorylation of tyrosine of p27, thereby disturbing the kinase activity of CDK4 and inhibiting cancer cell progression into the cell cycle.

Alt-Brk is an ALTternatively-spliced form of Brk containing the SH3 domain, which blocks pY88 and acts as an endogenous CDK4 inhibitor, and therefore was identified as a targetable regulatory region within p27. Brk is overexpressed in 60% of breast carcinomas, suggesting that it facilitates cell cycle progression by modulating CDK4 through p27 tyrosine phosphorylation. Phosphorylation of Tyr-88/Tyr-89 in the 310 helix of p27 and possibly Y74 reduces its cyclin-dependent kinase (CDK) inhibitory activity. This causes a conformational change in the p27-cyclin D-CDK4 complex, permitting p27 to vacate the catalytic cleft to allow ATP access and further phosphorylation of the active site. Thus, phosphorylation of this site can switch the tumor suppressive CDK inhibitory activity to an oncogenic activity.

Blocking CDK4 activity has long been a goal in cancer therapy. However, this has proven difficult due to the conservation between the active sites of serine/threonine kinases. Most inhibitors reacted with too many other essential kinases to provide any therapeutic benefit. Palbociclib is a CDK4 inhibitor, that appears to be extremely specific for CDK4 activity. The advantage of targeting p27 tyrosine (Y) phosphorylation as an indirect way to target CDK4 activity is that p27 has few substrates and as such its targeting should be more specific. Additionally, use of Palbociclib has shown that targeting CDK4 is a valid approach. The p27 tyrosine phosphorylation mimetic provides an additional approach for targeting this important kinase, which may have additional benefits.

In one aspect, the peptide increases cancer cells reactive oxygen species (ROS) level.

Reactive oxygen species (ROS) are highly reactive chemicals formed from diatomic oxygen (O₂), water, and hydrogen peroxide. Some prominent ROS are hydroperoxide (O₂H), superoxide (O₂—), [1] hydroxyl radical (OH·), and singlet oxygen.

ROS are byproducts of the normal metabolism of oxygen. ROS have roles in cell signaling and homeostasis, and are intrinsic to cellular functioning, are present at low and stationary levels in normal cells. ROS can cause irreversible damage to DNA as they oxidize and modify some cellular components and prevent them from performing their original functions. This suggests that ROS have a dual role; whether they will act as harmful, protective or signaling factors depends on the balance between ROS production and disposal at the right time and place. In other words, oxygen toxicity can arise both from uncontrolled production and from the inefficient elimination of ROS by the antioxidant system. During times of environmental stress (e.g., UV or heat exposure), ROS levels can increase dramatically. This may result in significant damage to cell structures. Cumulatively, this is known as oxidative stress. The production of ROS is strongly influenced by stress factor responses in plants, these factors that increase ROS production include drought, salinity, chilling, defense of pathogens, nutrient deficiency, metal toxicity and UV-B radiation. ROS are also generated by exogenous sources such as ionizing radiation generating irreversible effects in the development of tissues. If too much damage is present in mitochondria, a cell undergoes apoptosis or programmed cell death.

In general, the harmful effects of reactive oxygen species on the cell are the damage of DNA or RNA, oxidation of polyunsaturated fatty acids in lipids (lipid peroxidation), oxidation of amino acids in proteins, and oxidative deactivation of specific enzymes by oxidation co-factors. ROS are constantly generated and eliminated in the biological system and are required to drive regulatory pathways. Under normal physiological conditions, cells control ROS levels by balancing the generation of ROS with their elimination by scavenging systems. But under oxidative stress conditions, excessive ROS can damage cellular proteins, lipids and DNA, leading to fatal lesions in the cell that contribute to carcinogenesis. Cancer cells exhibit greater ROS stress than normal cells do, partly due to oncogenic stimulation, increased metabolic activity and mitochondrial malfunction. ROS is a double-edged sword. On one hand, at low levels, ROS facilitates cancer cell survival since cell-cycle progression driven by growth factors and receptor tyrosine kinases (RTK) require ROS for activation and chronic inflammation, a major mediator of cancer, is regulated by ROS. On the other hand, a high level of ROS can suppress tumor growth through the sustained activation of cell-cycle inhibitor and induction of cell death as well as senescence by damaging macromolecules. In fact, most of the chemotherapeutic and radiotherapeutic agents kill cancer cells by augmenting ROS stress. The ability of cancer cells to distinguish between ROS as a survival or apoptotic signal is controlled by the dosage, duration, type, and site of ROS production. Modest levels of ROS are required for cancer cells to survive, whereas excessive levels kill them.

The methods described herein are for use for the treatment of cancer. Cancer is a group of diseases involving abnormal cell growth with the potential to invade or spread to other parts of the body. In 2015, about 90.5 million people had cancer, about 14.1 million new cases occur a year and it caused about 8.8 million deaths (15.7% of deaths). The most common types of cancer in males are lung cancer, prostate cancer, colorectal cancer and stomach cancer. In females, the most common types are breast cancer, colorectal cancer, lung cancer and cervical cancer.

The term “cancer” refers to a group of diseases characterized by abnormal and uncontrolled cell proliferation starting at one site (primary site) with the potential to invade and to spread to other sites (secondary sites, metastases) which differentiate cancer (malignant tumor) from benign tumor. Virtually all the organs can be affected, leading to more than 100 types of cancer that can affect humans. Cancers can result from many causes including genetic predisposition, viral infection, exposure to ionizing radiation, exposure environmental pollutant, tobacco and or alcohol use, obesity, poor diet, lack of physical activity or any combination thereof. As used herein, “neoplasm” or “tumor” including grammatical variations thereof, means new and abnormal growth of tissue, which may be benign or cancerous. In a related aspect, the neoplasm is indicative of a neoplastic disease or disorder, including but not limited, to various cancers. For example, such cancers can include prostate, pancreatic, biliary, colon, rectal, liver, kidney, lung, testicular, breast, ovarian, pancreatic, brain, and head and neck cancers, melanoma, sarcoma, multiple myeloma, leukemia, lymphoma, and the like.

Exemplary cancers described by the national cancer institute include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood', Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor.

In one aspect, the cancer is pancreatic cancer. Pancreatic cancer arises when cells in the pancreas, the glandular organ behind the stomach, begin to multiply out of control and form a mass.

The most common, pancreatic adenocarcinoma, accounts for about 90% of cases. These adenocarcinomas start within the part of the pancreas that produces digestive enzymes, known as the exocrine component. Those cancers are often referred to as exocrine pancreatic cancers. Several other types of cancer, which collectively represent the majority of the non-adenocarcinomas, can also arise from these cells.

About 1-2% of cases of pancreatic cancer are neuroendocrine tumors, which arise from the hormone-producing cells of the pancreas. These are generally less aggressive than pancreatic adenocarcinoma. The small minority of pancreatic cancers that arise in the hormone-producing (endocrine) tissue of the pancreas have different clinical characteristics and are called pancreatic neuroendocrine tumors, sometimes abbreviated as “PanNETs”. Both groups occur mainly (but not exclusively) in people over 40, and are slightly more common in men, but some rare subtypes mainly occur in women or children.

The exocrine group is dominated by pancreatic adenocarcinoma. Nearly all these start in the ducts of the pancreas, as pancreatic ductal adenocarcinoma (PDAC). This is despite the fact that the tissue from which it arises—the pancreatic ductal epithelium—represents less than 10% of the pancreas by cell volume, because it constitutes only the ducts (an extensive but capillary-like duct-system fanning out) within the pancreas. This cancer originates in the ducts that carry secretions (such as enzymes and bicarbonate) away from the pancreas. About 60-70% of adenocarcinomas occur in the head of the pancreas. The next-most common type, acinar cell carcinoma of the pancreas, arises in the clusters of cells that produce these enzymes, and represents 5% of exocrine pancreas cancers. Like the ‘functioning’ endocrine cancers, acinar cell carcinomas may cause over-production of certain molecules, in this case digestive enzymes, which may cause symptoms such as skin rashes and joint pain. Cystadenocarcinomas account for 1% of pancreatic cancers, and they have a better prognosis than the other exocrine types. Pancreatoblastoma is a rare form, mostly occurring in childhood, and with a relatively good prognosis. Other exocrine cancers include adenosquamous carcinomas, signet ring cell carcinomas, hepatoid carcinomas, colloid carcinomas, undifferentiated carcinomas, and undifferentiated carcinomas with osteoclast-like giant cells. Solid pseudopapillary tumor is a rare low-grade neoplasm that mainly affects younger women, and generally has a very good prognosis. Pancreatic mucinous cystic neoplasms are a broad group of pancreas tumors that have varying malignant potential.

The small minority of tumors that arise elsewhere in the pancreas are mainly pancreatic neuroendocrine tumors (PanNETs). Neuroendocrine tumors (NETs) are a diverse group of benign or malignant tumors that arise from the body's neuroendocrine cells, which are responsible for integrating the nervous and endocrine systems. NETs can start in most organs of the body, including the pancreas, where the various malignant types are all considered to be rare. PanNETs are grouped into ‘functioning’ and ‘nonfunctioning’ types, depending on the degree to which they produce hormones. The functioning types secrete hormones such as insulin, gastrin, and glucagon into the bloodstream, often in large quantities, giving rise to serious symptoms such as low blood sugar, but also favoring relatively early detection. The most common functioning PanNETs are insulinomas and gastrinomas, named after the hormones they secrete. The nonfunctioning types do not secrete hormones in a sufficient quantity to give rise to overt clinical symptoms, so nonfunctioning PanNETs are often diagnosed only after the cancer has spread to other parts of the body.

Pancreatic cancer management and treatment includes surgery (for resectable tumors), chemotherapy, and/or radiotherapy. Whether or not surgical resection can be offered depends on how much the cancer has spread, the exact location of the tumor (how it relates to the major blood vessels passing close to the pancreas. i.e., the precise extent of local anatomical adjacency to, or involvement of, the venous or arterial blood vessels), and on the general health of the patient (with careful consideration of projected post-operative recovery). After surgery, adjuvant chemotherapy with gemcitabine or 5-FU can be offered if the person is sufficiently fit, and after a recovery period. In people not suitable for curative surgery, chemotherapy may be used to extend life or improve its quality. Before surgery, neoadjuvant chemotherapy or chemoradiotherapy may be used in cases that are considered to be “borderline resectable” in order to reduce the cancer to a level where surgery could be beneficial. Chemotherapy using gemcitabine alone was the standard for about a decade, as a number of trials testing it in combination with other drugs failed to demonstrate significantly better outcomes. However, the combination of gemcitabine with erlotinib was found to increase survival modestly, and erlotinib was licensed by the FDA for use in pancreatic cancer in 2005. The FOLFIRINOX chemotherapy regimen using four drugs was found more effective than gemcitabine, but with substantial side effects, and is thus only suitable for people with good performance status. This is also true of protein-bound paclitaxel (nab-paclitaxel), which was licensed by the FDA in 2013 for use with gemcitabine in pancreas cancer. By the end of 2013, both FOLFIRINOX and nab-paclitaxel with gemcitabine were regarded as good choices for those able to tolerate the side-effects, and gemcitabine remained an effective option for those who were not. A head-to-head trial between the two new options is awaited, and trials investigating other variations continue. The role of radiotherapy as an auxiliary (adjuvant) treatment after potentially curative surgery has been controversial since the 1980s. Radiotherapy may form part of treatment to attempt to shrink a tumor to a resectable state, but its use on unresectable tumors remains controversial as there are conflicting results from clinical trials.

In various aspects, the pancreatic cancer is pancreatic ductal adenocarcinoma (PDAC).

In some aspects, PDAC is resistant to CDK4/6 inhibition due to activation of CDK2; thus in some aspects, inhibition of both CDK4/6 and CDK2 is used.

Cyclin-dependent kinase 4/6 (CDK4/6)-specific inhibitors, such as palbociclib (PD), have shown clinical efficacy, but primary or secondary resistance remains a problem. The G1-S phase cell-cycle transition is governed by two cyclin-cdk complexes, cyclin D-cdk4/6 and cyclin E-cdk2. Cyclin D-cdk4 (D-K4) phosphorylates the G1 gatekeeper Rb, causing the release of S-phase-specific transcription factors, such as E2F. E2F causes the transcriptional induction of cyclin E, which in turn partners with cdk2 to further phosphorylate Rb and irreversibly cause the transition into S-phase. Cyclin D1 and cdk4 are overexpressed in a variety of human cancers, and, in mouse models, loss of either prevents the development of certain oncogene-driven tumors. Targeting cdk4 activity has been a long-standing goal in the oncology field and, because D-K4 is downstream of most oncogenic signaling pathways, targeting this kinase might prevent the resistance that frequently occurs when cell surface or upstream signal transducers are inhibited. The advent of cdk4-specific inhibitors (cdk4i), such as PD, abemaciclib, or ribociclib, has demonstrated that cdk4 is a promising target. In combination with letrozole, PD extended median progression-free survival (PFS) for metastatic breast cancer patients from 10.2 to 20.2 months. However, the overall survival (OS) of patients treated with PD mirrored that seen in patients treated with letrozole alone, suggesting that resistance to this combination therapy occurs. In tissue culture lines, PD- or ribociclib-mediated arrest did not appear durable.

Cyclin D is a transcriptional target of the MAPK pathway, but after cyclin D partners with cdk4, the dimer is unstable, and rapidly dissociates back into the monomeric forms, unless a third protein, p27 (also known as p27Kip1) or p21Cip1, holds the complex together. However, p27 binds to D-K4 in two different conformations: a closed and inactivating conformation or an open and activating form. This transition is mediated by the tyrosine (Y) phosphorylation of p27 on residues Y88 (or Y89). Non-phosphorylated p27-D-K4 complexes are catalytically inactive because the associated p27 blocks the ATP-binding site on cdk4 and prevents the required cdk activating kinase phosphorylation of the cdk4 domain itself. p27 Y88 is phosphorylated by Brk (breast tumor related kinase or PTK6, protein tyrosine kinase 6).

Brk, like other kinases in the Src family, share a domain organization comprising a tyrosine kinase domain (also termed SH1) plus one each of the protein-protein interaction domains SH2 and SH3, which bind to phosphotyrosine and proline-rich sequences (PXXP), respectively. The SH2 and SH3 domains recognize specific amino acid sequences within itself, thus is capable of adopting an autoinhibited state. Upon release from this inhibition by upstream signaling molecules, the SH2 and SH3 domains are free to bind downstream target proteins. Brk binds more strongly to p27 than the rest of the Src family of kinases and permits phosphorylation at Y88 p27. Overexpression of a naturally occurring alternatively spliced form of Brk (ALT), which contains the SH3 binding domain for p27, but lacks the SH1 kinase domain, inhibits phosphorylation of p27, inhibits cdk4, and causes growth arrest, suggesting that inhibition of p27 Y88 phosphorylation might be an alternative way to target cdk4-dependent tumors.

In contrast to cdk4, cdk2 does not require p27 to stabilize its interaction with cyclin E, rather cdk2 phosphorylation of RB is inhibited whenever p27, phosphorylated or not phosphorylated, is associated with the complex. This complex retains the ability to phosphorylate p27 on residue T187, and results in decreased p27 stability because it becomes a target for ubiquitin-mediated degradation. Reducing p27 association with cdk2, thus indirectly activates the cyclin E-cdk2 complex. Thus, blocking pY88 has the added benefit of preventing p27 degradation since p27 is stabilized in the non-phosphorylated form which inhibits cdk2 as well as cdk4. When resistance to cdk4 inhibiting therapies occurs, such as observed with PD (and PD plus letrozole), the cells can compensate for the loss of cdk4 activity with cdk2, so a therapy that inhibits both kinases at the outset might offer therapeutic advantages.

In one aspect, the pharmaceutical composition further includes a delivery vehicle.

The delivery vehicle is a system compatible with the other ingredients of the formulation, and not deleterious to the recipient thereof, nor to the activity of the active ingredient of the formulation, that is used to successfully address delivery-related problems, to carry the peptide of interest to the desired sites of therapeutic action while reducing adverse side effects, and to allow its efficient penetration inside the target cell.

In some aspects, the delivery vehicle is selected from the group consisting of a nanoparticle, a liposome, a dendrimer, a micelle, a nanoemulsion, a nanosuspension, a niosome, a nanocapsule, a magnetic nanoparticle, a lipoprotein-based carrier, and a lipoplex nanoparticle.

The term “nanoparticle is used to define particle of matter that is between 1 and 150 nanometers (nm) in diameter. Nanoparticles occur in a great variety of shapes, which have been given many informal names such as nanospheres, nanorods, nanochains, nanostars, nanoflowers, nanoreefs, nanowhiskers, nanofibers, and nanoboxes. The shapes of nanoparticles may be determined by the intrinsic crystal habit of the material, or by the influence of the environment around their creation. Medicinal application of nanoparticle involves silver, gold, hydroxyapatite, clay, titanium dioxide, silicon dioxide, zirconium dioxide, carbon, diamond, aluminum oxide and ytterbium trifluoride as the base material. Semi-solid and soft nanoparticles such as liposome can also be generated. Various types of liposome nanoparticles are currently used clinically as delivery systems for anticancer drugs and vaccines.

“Nanocapsule” refers to a thin membrane surrounding a core (liquid, solid) and having a size ranging from 10 nm to 1000 nm. Nanocapsules are submicroscopic colloidal drug carrier systems composed of an oily or an aqueous core surrounded by a thin polymer membrane, which may be composed of natural or synthetic polymers.

A “magnetic nanoparticle” is a nanoparticle having magnetic core with a polymer or metal coating which can be functionalized or may consist of porous polymers that contain magnetic nanoparticles precipitated within the pores. By functionalizing the polymer or metal coating it is possible to attach, for example, cytotoxic drugs for targeted chemotherapy or therapeutic peptide. Once attached, the particle/therapeutic agent complex is injected into the bloodstream, often using a catheter to position the injection site near the target. Magnetic fields, generally from high-field, high-gradient, rare earth magnets are focused over the target site and the forces on the particles as they enter the field allow them to be captured and extravasated at the target.

The term “liposome” or “lipoplex nanoparticle” refers to non-toxic, non-hemolytic, and non-immunogenic lipid-based, ligand-coated nanocarriers that can store their payload in the hydrophobic shell or the hydrophilic interior depending on the nature of the drug/contrast agent being carried. Liposomes are biocompatible and biodegradable and can be designed to avoid clearance mechanisms (reticuloendothelial system (RES), renal clearance, chemical or enzymatic inactivation, etc.). Polyethylene glycol (PEG) can be added to the surface of the liposomes to increase their relatively low stability in vitro; PEGylation of the liposomal nanocarrier elongates the half-life of the construct while maintaining the passive targeting mechanism that is commonly conferred to lipid-based nanocarriers.

Other lipid-based nanocarriers include nanoemulsion and lipoprotein-based carrier. As used herein, the term “nanoemulsion” refers to a colloidal system consisting of mainly oil, surfactant, and water, and having a high kinetic stability, low viscosity. Non limiting examples of oils that can be used in the formation of nanoemulsion include castor oil, corn oil, coconut oil, evening primrose oil, linseed oil, mineral oil, olive. Emulgent such as natural lecithins from plant or animal source, phospholipids, castor oil can be part of the composition of the nanoemulsion. Non-limiting examples of surfactant or co-surfactant include polysorbate20, polysorbate80, polyoxy60, castor oil, sorbitan monooleate, ethanol, glycerin, PEG300, PEG400, polyene glycol, and poloxamer. Lipoprotein-based carrier include lipoproteins, which are biological lipid carriers playing important role in transport of fats within the body. These are natural nanoparticles which serve as drug-delivery vehicles due to their small size, long residence time in the circulation. Examples of lipoprotein include low-density lipoprotein (LDL), which carries cholesterol in plasma. Lipoproteins carry high-drug payload and are used as delivery vehicles for transportation of chemotherapeutic agents.

As used herein, “dendrimer” and “micelle” can be used interchangeably and refer to polymeric based delivery vehicles. Polymeric micelles can be prepared from certain amphiphilic co-polymers consisting of both hydrophilic and hydrophobic monomer units. Dendrimers have a core that branches out in regular intervals to form a small, spherical, and very dense nanocarrier.

A “nanosuspension”, as used herein consists of a pure poorly water-soluble drug without any matrix material suspended in dispersion. Preparation of nanosuspension is simple and applicable to all drugs which are water insoluble.

As used herein, “niosome” refers to a drug delivery vehicle including nonionic surfactants capable of entrap hydrophilic and lipophilic compound. Niosomes are vesicles composed of non-ionic surfactants, which are biodegradable, relatively nontoxic, more stable and inexpensive, an alternative to liposomes. The properties of the vesicles can be changed by varying the composition of the vesicles, size, lamellarity, tapped volume, surface charge and concentration.

In various aspects, the lipoplex nanoparticle includes 1,2-di-O-octdecenyl-3-trimethyl ammonium propane (DOTMA), cholesterol, DOPE, TPGS, or a combination thereof. In other aspects, the lipoplex nanoparticle includes DOTMA and cholesterol. In other aspects, the lipoplex nanoparticle includes DOTMA, cholesterol and TPGS.

In other aspects, the lipoplex nanoparticle includes DOTMA and cholesterol at a molar ratio from about 10:90 to 90:10. In some aspects, the lipoplex nanoparticle includes DOTMA and cholesterol at a molar ratio from about 40:50 to 50:39. In one aspect, the lipoplex nanoparticle includes DOTMA, cholesterol and TPGS at a molar ratio of about 50:49:1.

In one aspect, a lipid to peptide mass ratio is about 8:1, about 6:1, 5:1, about 10:1, about 12.5:1, about 15:1, about 20:1, about 25:1, about 30:1 or about 35:1. In some aspects, the lipid to peptide mass ratio is about 10:1. In a preferred embodiment, the lipid to peptide mass ratio is about 12.5:1.

The isolated peptide of the invention can be encapsulated using various nanoparticle formulations, which maintain the properties of the peptide (i.e., CDK4 and CDK2 dual inhibition for efficient use as a breast cancer therapy).

The use of lipoplex nanoparticle for the liposomal encapsulation of the peptide can for example include a combination of DOTMA, cholesterol, DOPE, and TPGS.

Useful cationic lipids with respect to the present invention include but are not limited to: DDAB, dimethyldioctadecyl ammonium bromide: N-1-(2,3-dioloyloxy)propyl-N,N,N-trimethyl ammonium methylsulfate; 1,2-diacyloxy-3-trimethylammonium propanes, (including but not limited to, dioleoyl (DOTAP), dilauroyloxy, dimyristoyloxy, dipalmitoyloxy, and distearoyloxy); N-1-(2,3-dioleoyloxy)propyl-N,N-dimethyl amine; 1,2-diacyl-3-dimethylammonium propanes, (including but not limited to, dioleoyl (DODAP), dilauroyl. dimyristoyl, dipalmitoyl, and distearoyl); DOTMA, N-1-2,3-bis(oleyloxy)propyl-N,N,N-trimethylammonium chloride, (including but not limited to, dioleyl (DOTMA), dilauryl, dimyristyl, dipalmityl, and distearyl); DOGS, dioctadecylamidoglycylspermine; DC-cholesterol, 3 B—N(N′,N′dimethylaminoethane) carbamoylcholesterol: DOSPA, 2,3-dioleoyloxy-N-(2-(sperminecarboxamido)-ethyl)-N,N-dimethyl-1-propanaminium trifluoroacetate; 1,2-diacyl-sn-glycero-3-ethylphosphocholines (including but not limited to dioleoyl (DOEPC), dilauroyl, dimyristoyl, dipalmitoyl, distearoyl, and palmitoyl-oleoyl); B-alanyl cholesterol: CTAB, cetyl trimethyl ammonium bromide: diC14-amidine, N-t-butyl-N′-tetradecyl-3-tetradecylaminopropionamidine; 14 Dea2; TMAG, N-(alpha-trimethylammonioacetyl)didodecyl-D-glutamate chloride; O,O′-ditetradecanoyl-N-(trimethylammonioacetyl) diethanolamine chloride: DOSPER, 1,3-dioleoyloxy-2-(6-carboxy-spermyl)-propylamide; N,N.N′,N′-tetramethyl N,N′-bis(2-hydroxylethyl)-2,3-dioleoyloxy-1,4-butanediammonium iodide, 1-2-(acyloxy)ethyl-2-alkyl (alkenyl)-3-(2-hydroxyethyl) imidazolinium chloride, derivatives such as DOTIM, 1-2-(9(Z)-octadecenoyloxy)ethyl-2-(8(Z)-heptadecenyl-3-(2-hydroxyethyl) imidazolinium chloride; DPTIM, 1-2-(hexadecanoyloxy)ethyl)-2-pentadecyl-3-(2-hydroxyethyl) imidazolinium chloride; 2,3-dialkyloxypropyl quaternary ammonium compound derivatives, contain a hydroxyalkyl moiety on the quaternary amine such as: DORI, 1,2-dioleoyl-3-dimethyl-hydroxyethyl ammonium bromide: DORIE, 1,2-dioleyloxypropyl-3-dimethyl-hydroxyethyl ammonium bromide: DORIE-HP 1,2-dioley bromide: DORIE-HB, 1,2-dioleyloxypropyl-3-dimethyl hydroxybutyl ammonium bromide: DORIE-HPe, 1,2-dioleyloxypropyl-3-dimethyl-hydroxypentyl ammonium bromide; DMRIE, 1,2-dimyristyloxypropyl-3-dimethyl-5 hydroxylethyl ammonium bromide: DPRIE, 1,2-dipalmityloxypropyl-3-dimethyl-hydroxyethyl ammonium bromide; DSRIE, 1,2-disteryloxypropyl-3-dimethyl-hydroxyethyl ammonium bromide.

Other liposomes are included in this invention. For instance, one aspect of this present invention provides a liposome comprising between 25% and 45% (mol/mol) of an anionic lipid. The content of anionic lipid affects important characteristics of the liposome, such as lipid hydrolysis of the liposome and also the immune response toward the liposome. As the content of anionic lipid increases, so does the rate of lipid hydrolysis (and the release of drug). It has been demonstrated that a reasonable rate of hydrolysis can be achieved by anionic lipid content between 25% and 45%. Thus, in one embodiment, the content of anionic lipid is at least 25%. In another embodiment, the content of anionic lipid is no more than 45%. In yet another embodiment, the anionic lipid content of the liposome is selected from the group consisting of between 25% and 45%, 28% and 42%, 30% and 40%, 32% and 38% and 34% and 36%. The immune response toward the liposomes can be affected by the content of anionic lipid. Thus, the clearance rate of the liposome in body may be reduced by keeping the content of the anionic lipid in the liposome below a certain level and the content of anionic lipid in the liposome can be used to strike a balance between hydrolysis rate and clearance by the reticuloendothelial system.

Preferably the anionic lipid is a phospholipid and preferably, the phospholipid is selected from the group consisting of PI (phosphatidyl inositol), PS (phosphatidyl serine), DPG (bisphosphatidyl glycerol), PA (phosphatidic acid), PEOH (phosphatidyl alcohol), and PG (phosphatidyl glycerol). More preferably, the anionic phospholipid is PG.

In one aspect, the method further includes administering to the subject an anti-cancer treatment.

In some aspects administration can be in combination with one or more additional therapeutic agents. The phrases “combination therapy”, “combined with” and the like refer to the use of more than one medication or treatment simultaneously to increase the response. The composition of the present invention might for example be used in combination with other drugs or treatment in use to treat cancer. Specifically, the administration of the composition of the present invention to a subject can be in combination with any anti-cancer therapies. Such therapies can be administered prior to, simultaneously with, or following administration of the composition of the present invention.

In other aspects, the anti-cancer treatment is administered prior to, simultaneously with, or after administration of the pharmaceutical composition.

The term “anti-cancer therapy” or “anti-cancer treatment” as used herein is meant to refer to any treatment that can be used to treat cancer, such as surgery, radiotherapy, chemotherapy, immunotherapy, and checkpoint inhibitor therapy.

Examples of chemotherapy include treatment with a chemotherapeutic, cytotoxic or antineoplastic agents including, but not limited to, (i) anti-microtubules agents comprising vinca alkaloids (vinblastine, vincristine, vinflunine, vindesine, and vinorelbine), taxanes (cabazitaxel, docetaxel, larotaxel, ortataxel, paclitaxel, and tesetaxel), epothilones (ixabepilone), and podophyllotoxin (etoposide and teniposide); (ii) antimetabolite agents comprising anti-folates (aminopterin, methotrexate, pemetrexed, pralatrexate, and raltitrexed), and deoxynucleoside analogues (azacitidine, capecitabine, carmofur, cladribine, clofarabine, cytarabine, decitabine, doxifluridine, floxuridine, fludarabine, fluorouracil, gemcitabine, hydroxycarbamide, mercaptopurine, nelarabine, pentostatin, tegafur, and thioguanine); (iii) topoisomerase inhibitors comprising Topoisomerase I inhibitors (belotecan, camptothecin, cositecan, gimatecan, exatecan, irinotecan, lurtotecan, silatecan, topotecan, and rubitecan) and Topoisomerase II inhibitors (aclarubicin, amrubicin, daunorubicin, doxorubicin, epirubicin, etoposide, idarubicinm, merbarone, mitoxantrone, novobiocin, pirarubicin, teniposide, valrubicin, and zorubicin); (iv) alkylating agents comprising nitrogen mustards (bendamustine, busulfan, chlorambucil, cyclophosphamide, estramustine phosphate, ifosamide, mechlorethamine, melphalan, prednimustine, trofosfamide, and uramustine), nitrosoureas (carmustine (BCNU), fotemustine, lomustine (CCNU), N-Nitroso-N-methylurea (MNU), nimustine, ranimustine semustine (MeCCNU), and streptozotocin), platinum-based (cisplatin, carboplatin, dicycloplatin, nedaplatin, oxaliplatin and satraplatin), aziridines (carboquone, thiotepa, mytomycin, diaziquone (AZQ), triaziquone and triethylenemelamine), alkyl sulfonates (busulfan, mannosulfan, and treosulfan), non-classical alkylating agents (hydrazines, procarbazine, triazenes, hexamethylmelamine, altretamine, mitobronitol, and pipobroman), tetrazines (dacarbazine, mitozolomide and temozolomide); (v) anthracyclines agents comprising doxorubicin and daunorubicin. Derivatives of these compounds include epirubicin and idarubicin; pirarubicin, aclarubicin, and mitoxantrone, bleomycins, mitomycin C, mitoxantrone, and actinomycin; (vi) enzyme inhibitors agents comprising FI inhibitor (Tipifarnib), CDK inhibitors (Abemaciclib, Alvocidib, Palbociclib, Ribociclib, and Seliciclib), PrI inhibitor (Bortezomib, Carfilzomib, and Ixazomib), PhI inhibitor (Anagrelide), IMPDI inhibitor (Tiazofurin), LI inhibitor (Masoprocol), PARP inhibitor (Niraparib, Olaparib, Rucaparib), HDAC inhibitor (Belinostat, Panobinostat, Romidepsin, Vorinostat), and PIKI inhibitor (Idelalisib); (vii) receptor antagonist agent comprising ERA receptor antagonist (Atrasentan), Retinoid X receptor antagonist (Bexarotene), Sex steroid receptor antagonist (Testolactone); (viii) ungrouped agent comprising Amsacrine, Trabectedin, Retinoids (Alitretinoin Tretinoin) Arsenic trioxide, Asparagine depleters (Asparaginase/Pegaspargase), Celecoxib, Demecolcine Elesclomol, Elsamitrucin, Etoglucid, Lonidamine, Lucanthone, Mitoguazone, Mitotane, Oblimersen, Omacetaxine mepesuccinate, and Eribulin.

Examples of immunotherapy include treatment with antibodies including, but not limited to, alemtuzumab, Avastin (bevacizumab), Bexxar (tositumomab), CDP 870, and CEA-Scan (arcitumomab), denosumab, Erbitux (cetuximab), Herceptin (trastuzumab), Humira (adalimumab), IMC-IIF 8, LeukoScan (sulesomab), MabCampath (alemtuzumab), MabThera (Rituximab), matuzumab, Mylotarg (gemtuzumab oxogamicin), natalizumab, NeutroSpec (Technetium (99mTc) fanolesomab), panitumamab, Panorex (Edrecolomab), ProstaScint (Indium-Ill labeled Capromab Pendetide), Raptiva (efalizumab), Remicade (infliximab), ReoPro (abciximab), rituximab, Simulect (basiliximab), Synagis (palivizumab), TheraCIM hR3, tocilizumab, Tysabri (natalizumab), Verluma (nofetumomab), Xolair (omalizumab), Zenapax (dacliximab), Zevalin (ibritumomab tiuxetan (IDEC-Y2B8) conjugated to yttrium 90), Gilotrif (afatinib), Lynparza (olaparib), Perjeta (pertuzumab), Otdivo (nivolumab), Bosulif (bosutinib), Cabometyx (cabozantinib), trastuzumab-dkst (Ogivri), Sutent (sunitinib malate), Adcetris (brentuximab vedotin), Alecensa (alectinib), Calquence (acalabrutinib), Yescarta (ciloleucel), Verzenio (abemaciclib), Keytruda (pembrolizumab), Aliqopa (copanlisib), Nerlynx (neratinib), Imfinzi (durvalumab), Darzalex (daratumumab), Tecentriq (atezolizumab), and Tarceva (erlotinib).

In some aspects, the anti-cancer treatment is selected from the group consisting of chemotherapy, radiation treatment, immunotherapy, resection of a tumor, and any combination thereof.

In some aspects, the anti-cancer treatment is an anti-cancer agent selected from the group consisting of palbociclib, ribociclib, abemaciclib, osirmetinib, gefitinib, lapatinib, pantitumumab, vandetanib, necitumumab, vemurafenib, sorafenib tosylate, PLX-4720, dabrafenib, paclitaxel, cisplatin, docetaxol, carboplatin, vincristine, vinblastine, methotrexate, cyclophosphamide, CPT-11, 5-fluorouracil, gemcitabine, estramustine, carmustine, adriamycin, etoposide, arsenic trioxide, irinotecan, vemurafenib, erlotinib, cetuximab, letrozole, fulvestrant, and epolhilone derivatives.

In another embodiment, the invention provides a method of treating pancreatic cancer in a subject in need thereof including administering to the subject a therapeutically effective amount of a pharmaceutical composition including a cyclin-dependent kinase 4/6 (CDK4/6) and CDK2-specific inhibitor, thereby treating pancreatic cancer in the subject.

In one aspect, the CDK4/6 and CDK2-specific inhibitor is an isolated peptide having the amino acid sequence of SEQ ID NO:6.

In one aspect, the peptide inhibits cancer cell proliferation and/or decreases cancer cell viability. In another aspect, the peptide inhibits tumor growth. In one aspect, the peptide increases cancer cell death. In another aspect, the peptide increases tumor necrosis. In one aspect, the peptide increases cancer cells reactive oxygen species (ROS) level. In one aspect, the peptide inhibits phosphorylation of p27. In another aspect, the peptide inhibits CDK2 and CDK4.

In various aspects, the pancreatic cancer is pancreatic ductal adenocarcinoma (PDAC). In some aspects, the PDAC is cyclin-dependent kinase 4/6 (CDK4/6) inhibitor resistant.

In one aspect, the pharmaceutical composition further includes a delivery vehicle. In some aspects, the delivery vehicle is selected from the group consisting of a nanoparticle, a liposome, a dendrimer, a micelle, a nanoemulsion, a nanosuspension, a niosome, a nanocapsule, a magnetic nanoparticle, a lipoprotein-based carrier, and a lipoplex nanoparticle. In various aspects, the lipoplex nanoparticle includes 1,2-di-O-octdecenyl-3-trimethyl ammonium propane (DOTMA), cholesterol, DOPE, TPGS, or a combination thereof. In other aspects, the lipoplex nanoparticle includes DOTMA and cholesterol. In other aspects, the lipoplex nanoparticle includes DOTMA, cholesterol and TPGS.

In one aspect, the method further includes administering to the subject an anti-cancer treatment. In some aspects, the anti-cancer treatment is selected from the group consisting of chemotherapy, radiation treatment, immunotherapy, resection of a tumor, and any combination thereof. In other aspects, the anti-cancer treatment is administered prior to, simultaneously with, or after administration of the pharmaceutical composition. In some aspects, the anti-cancer treatment is an anti-cancer agent selected from the group consisting of palbociclib, ribociclib, abemaciclib, osirmetinib, gefitinib, lapatinib, pantitumumab, vandetanib, necitumumab, vemurafenib, sorafenib tosylate, PLX-4720, dabrafenib, paclitaxel, cisplatin, docetaxol, carboplatin, vincristine, vinblastine, methotrexate, cyclophosphamide, CPT-11, 5-fluorouracil, gemcitabine, estramustine, carmustine, adriamycin, etoposide, arsenic trioxide, irinotecan, vemurafenib, erlotinib, cetuximab, letrozole, fulvestrant, and epolhilone derivatives.

Presented below are examples discussing the use of the pharmaceutical composition described herein contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

### Truncated Alt Peptides

The truncated peptides used in the instant application were as follows.

The nomenclature of the peptides used in the instant application is either “CCL-x” or “IpY.x” without any difference in the meaning.

TABLE 1
peptides of the invention

| Name | Sequence | SEQ ID NO: |
|---|---|---|
| CCL-8 | MDYKDDDDKMVSRDQAHLGPKYVGLWDFKRSTDEELSFRAGDV | SEQ ID NO: 1 |
| | FHVARKEEQWWWATLLDEAGGAVAQGYVPHNYLAERETVESEP | |
| | AGHAGSAALQDLAASRGPAAPERGGLPQPARA | |
| CCL-9 | MDYKDDDDKMVSRDQAHLGPKYVGLWDFKSRTDEELSFRAGDV | SEQ ID NO: 2 |
| | FHVARKEEQWWWATLLDEAGGAVAQGYVPHNYLAERETVESEP | |
| | AGHAGCAALQDLAA | |
| CCL-10 | MDYKDDDDKMVSRDQAHLGPKYVGLWDFKSRTDEELSFRAGDV | SEQ ID NO: 3 |
| | FHVARKEEQWWWATLLDEAGGAVAQGYVPHNYLAERETVES | |
| CCL-14 | MVSRDQAHLGPKYVGLWDFKSRTDEELSFRAGDVFHVARKEEQ | SEQ ID NO: 4 |
| | WWWATLLDEAGGAVAQGYVPHNYLAERETVESEPAGHAGS | |
| CCL-19 | MVSRDQAHLGPKYVGLWDFKSRTDEELSFRAGDVFHVARKEEQ | SEQ ID NO: 5 |
| | WWWATLLDEAGGAVAQGYVPHNYLAERETVESEPAGHAGCAA | |
| | LQDLAA | |
| CCL-20 | MVSRDQAHLGPKYVGLWDFKSRTDEELSFRAGDVFHVARKEEQ | SEQ ID NO: 6 |
| | WWWATLLDEAGGAVAQGYVPHNYLAERETVESEPAGHAGSAAL | |
| | QDLAA | |
| CCL-21 | RRRRRRRRRMVSRDQAHLGPKYVGLWDFKSRTDEELSFRAGDVF | SEQ ID NO: 7 |
| | HVARKEEQWWWATLLDEAGGAVAQGYVPHNYLAERETVESEPA | |
| | GHAGSAALQDLAA | |

### Example 2

### Pancreatic Cancer Cell Lines are Resistant to Palbociclib

A panel of 8 Pancreatic Ductal Adenocarcinoma (PDAC) cell lines with KRAS and CDKN2A/p16 mutation were chosen. Despite the existence of mutations that affect CDK4/6 activity in some aspects, all lines were resistant to the CDK4/6i, palbociclib.

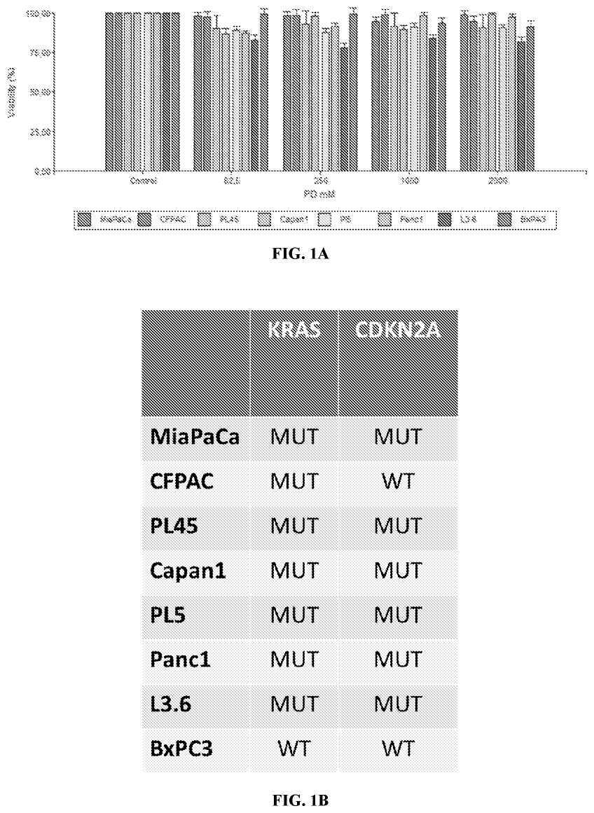

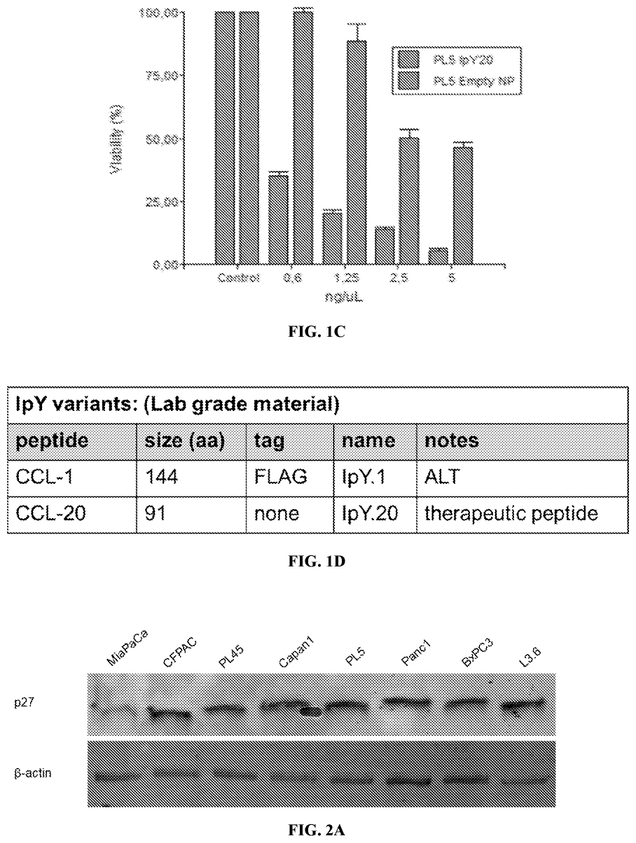

To test whether PDAC cells might be resistant to CDK4/6 inhibition due to activation of CDK2, experiments were performed. To determine whether inhibition of p27 Y phosphorylation by ALT treatment and the subsequent dual inhibition of CDK4/6 and CDK2 would inhibit PDAC cells, a smaller and more stable variant of ALT called IpY.20 was used. IpY.20 behaved similarly to IpY.1 in terms of its ability to arrest CDK4i-resistant and treatment naive breast cancer cells. To rule out effects from the liposome, cells were treated with empty NP as well. Some non-specific liposome toxicity was detected at the high concentrations. IpY.20 was then tested in the full PDAC panel (see FIGS. 1A-1D).

### Example 3

### P27 Expression and ROS Response

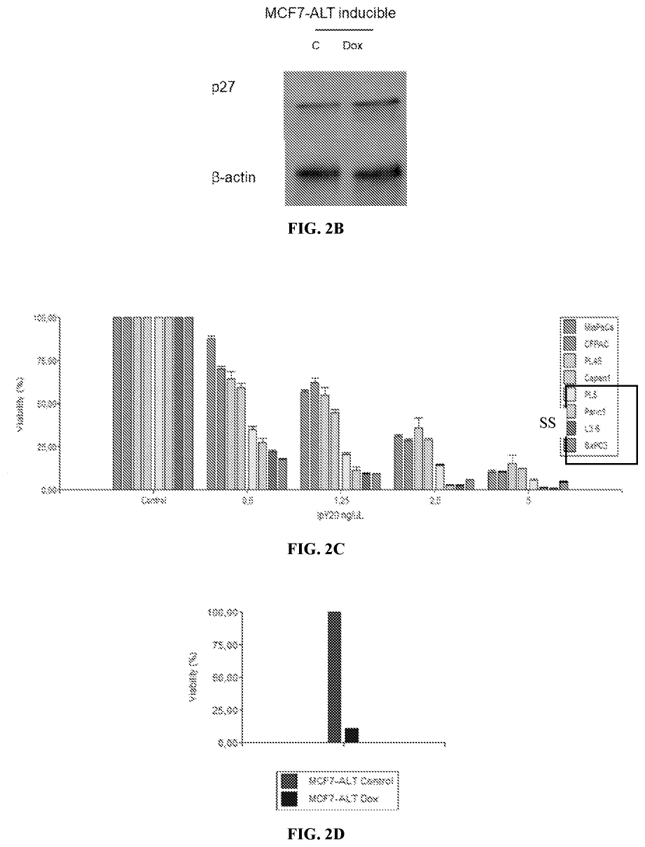

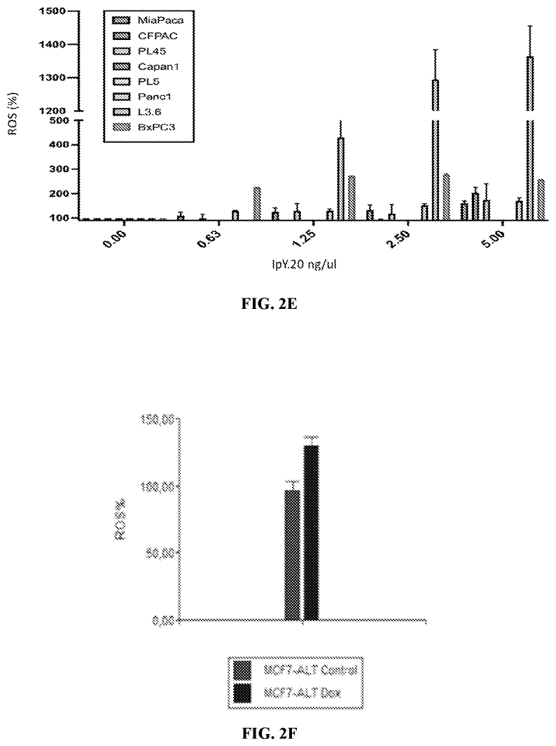

As illustrated in FIGS. 2A-2F, it was demonstrated that pancreatic cancer cell lines express p27 and are inhibited by IpY.20 treatment. It was also demonstrated that ROS is induced in the presence of IpY.20

### Example 4

Ip Y.20 Induces Necroptosis, which is Rescued by NEC-1 and NAC Treatment

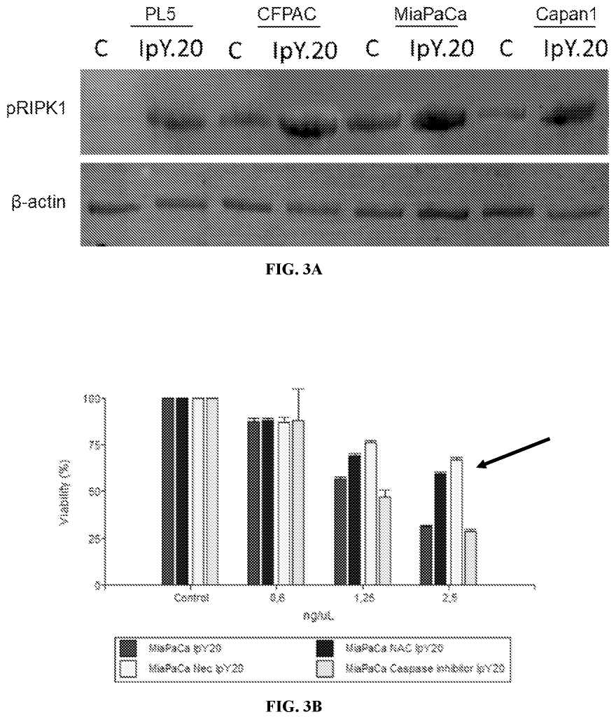

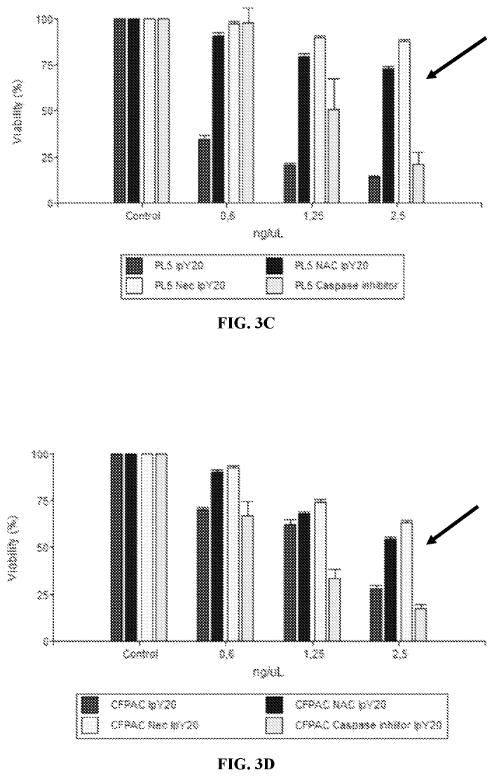

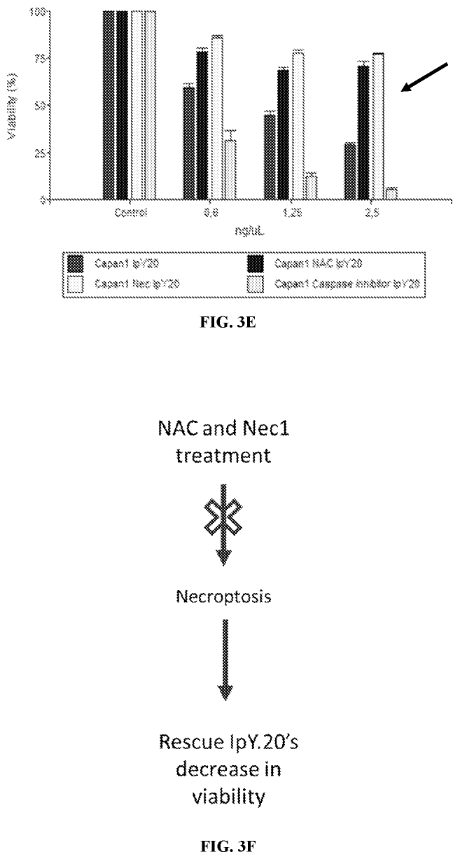

When the less sensitive PDAC lines were treated with IpY.20, pRIPK1 expression, a marker of necroptosis, increased (FIG. 3A). To determine if ROS-dependent induction of pRIPK1 and necroptosis was responsible for IpY.20's effect in PDAC cell lines, cells lines (A-D) were treated with N-acetyl-1-cysteine (5 mM) a ROS inhibitor, Necrostatin-1 (10 μM) which prevents RIPK1 phosphorylation and Caspase-3/7 Inhibitor, for 24 h, followed by IpY.20 for the next 24 h. N=4 *p<0.05. PDAC cell lines that increased expression of pRIPK1 after IpY.20 treatment were rescued by NAC and Nec1 pretreatment, suggesting that IpY.20's effect was dependent on ROS and necroptosis induction. Pretreatment of cells with the caspase inhibitor did not rescue cells (see FIGS. 3A-3F).

### Example 5

### IpY20 Induces Apoptosis

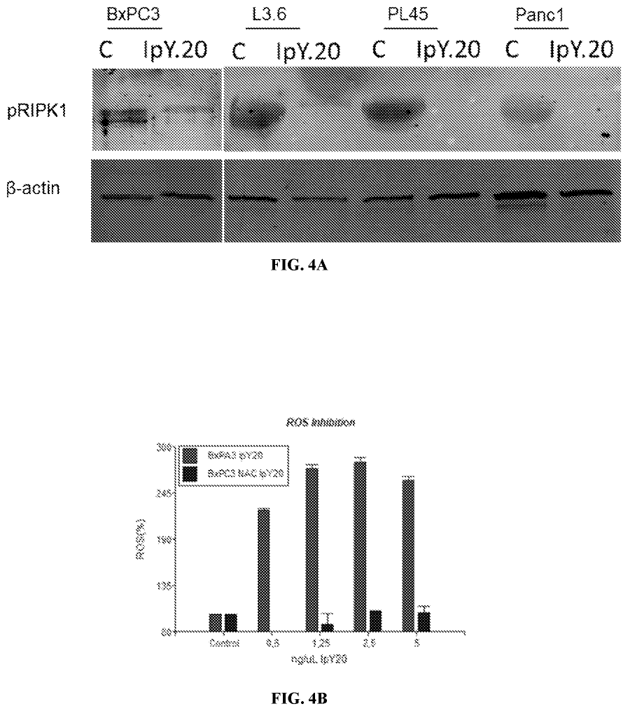

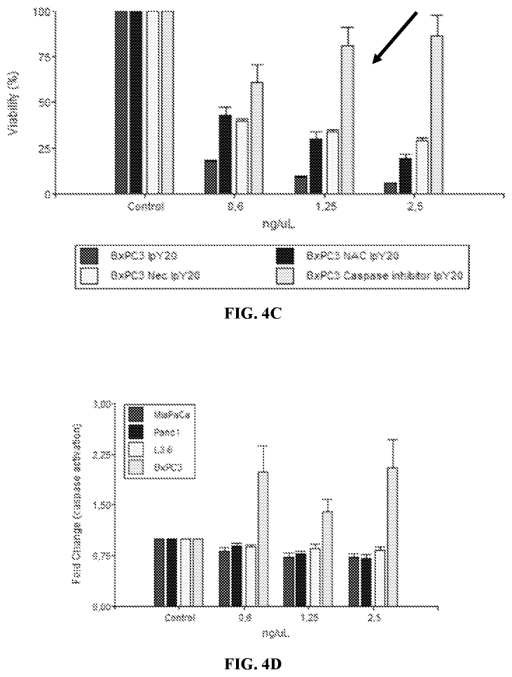

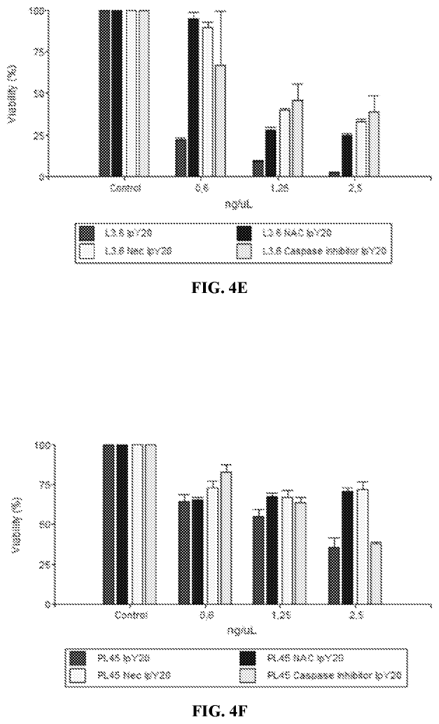

The Super sensitive PDAC lines did not exhibit induction of pRIPK1 after IpY.20 treatment (FIG. 4A). When these lines were pre-treated with NAC, Nec-1, or Caspase 3/7 inhibitor followed by IpY.20 treatment, differential results were observed. N=4 *p<0.05 (FIGS. 4A-4D). NAC treatment of BxPC3 cells reduced ROS levels (FIG. 4B), but these cells were rescued by caspase treatment (FIG. 4C). Cell lines were treated IpY.20 for 24 h and assayed for apoptosis using the Caspase-Glo 3/7 Assay and BxPC3 strongly induced caspase expression. Percent viability relative to control plotted in all cases. L3.6 (FIG. 4E), PL45 (FIG. 4F) and Panel (FIG. 4G) were rescued by NEC-1, NAC and caspase inhibition to differing extents. Biomarkers dictating response were determined.

### Example 6

### Drug Uptake Dictates IPY Sensitivity

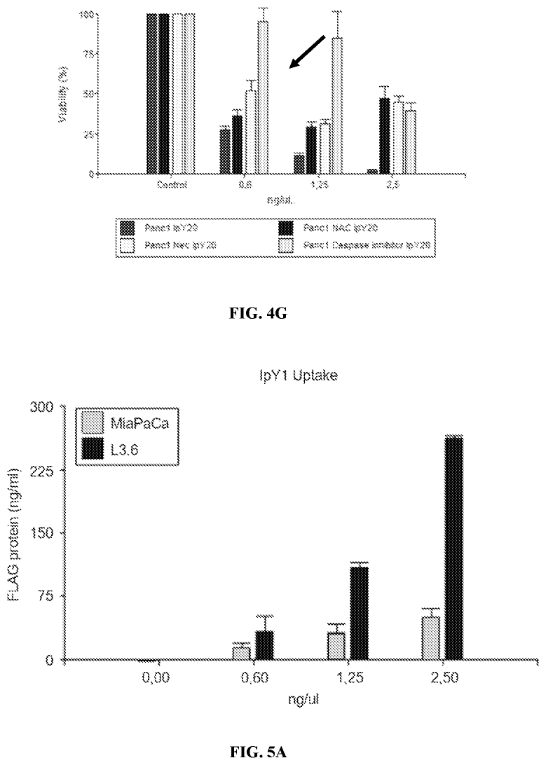

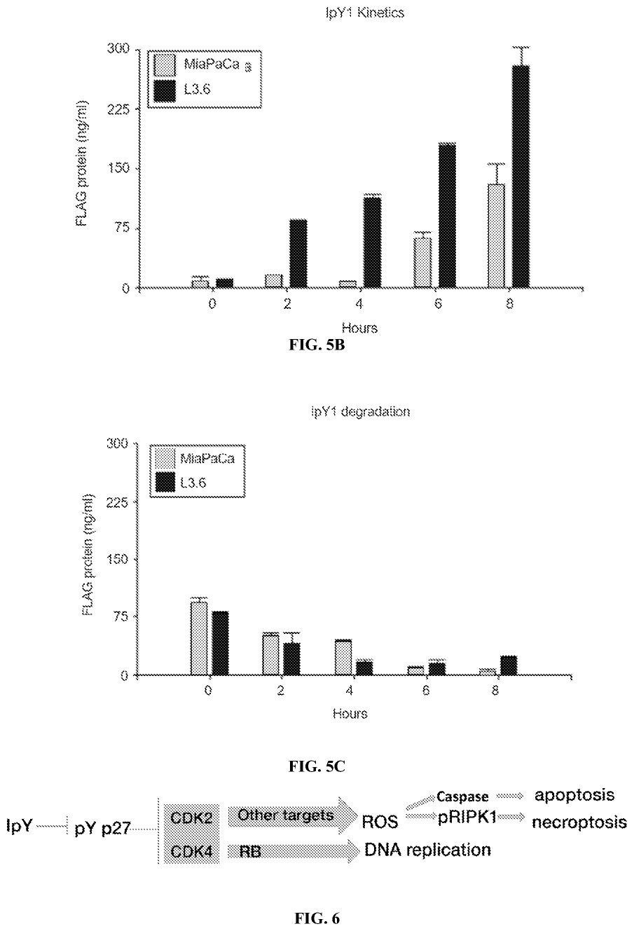

Different uptake kinetics of the liposomal IpY.20 might cause the different drug sensitivity. To examine this, IpY.1 was used, which has a FLAG tag-ALT and used a FLAG ELISA assay to determine the concentration of peptide delivered to cells at different concentrations and with time observed in the different PDAC lines. The Super sensitive line, L3.6, accumulated a higher concentration of peptide relative to the amount seen in MiaPaCa cells, suggesting that this was responsible in part for the increased sensitivity of this line. Biomarkers of uptake were determined (see FIGS. 5A-5C).

### Example 7

### ALT (PY.1) Causes Tumor Regression and Increases Overall Survival in a Palbociclib-Resistant Er+Breast Cancer Mouse Model

As the 144 aa ALT peptide does not enter cells, packaged ALT was formulated within a lipid nanoparticle (IpY.1) to facilitate delivery into tumor cells. MCF7 cells were treated with ALT peptide (FIG. 8, second-fourth from left), empty NP (FIG. 8, middle) or the assembled IpY.1 (FIG. 8, right) before analysis for proliferation.

IpY.1 causes tumor regression and increases overall survival in palbociclib-resistant tumors: MCF7 xenographs were rendered palbociclib-resistant by treatment with 0.75 mg/kg palbociclib for extended periods of time. When tumors≥700 mm³ in the presence of palbociclib, mice were randomly assigned to three different groups: (1) continued palbociclib (75 mg/kg, oral gavage, daily) (FIG. 9, second from left); (2) IpY.1 (0.75 mg/kg, i.v., 3×/week) (FIG. 9, third from left); (3) palbociclib+IpY.1 (FIG. 9, right). Tumor volumes were measured by calibers. Study endpoint: Tumor volumes>1500 mm³ or death.

### Example 8

### Conclusions

IpY.20 inhibits palbociclib-resistant PDAC cells, suggesting that inhibition of p27 pY and CDK4/6 and CDK2 may be an effective approach to treat PDAC. Differential sensitivity was observed, with some cell lines exhibiting Super sensitivity (SS) (PL5, Panc1, L3.6, and BXPC3).

Inhibition of p27 pY, CDK4/6 and CDK2 resulted in ROS induction, necroptosis and/or apoptosis, demonstrating a different MOA compared to CDK4/6i, where cell death is not observed. ROS was induced to different levels in all cell lines. IpY.20 induced necroptosis, as measured by pRIPK1 expression, and rescue by pre-treatment with NEC-1 and NAC was seen in the less sensitive PDAC lines. However, in the SS lines, both necroptosis and apoptosis appeared to be induced. Biomarkers of response are determined (see FIG. 6).

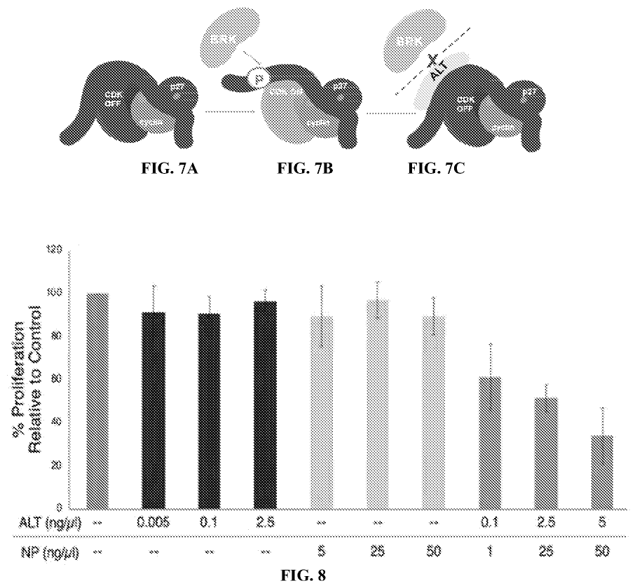

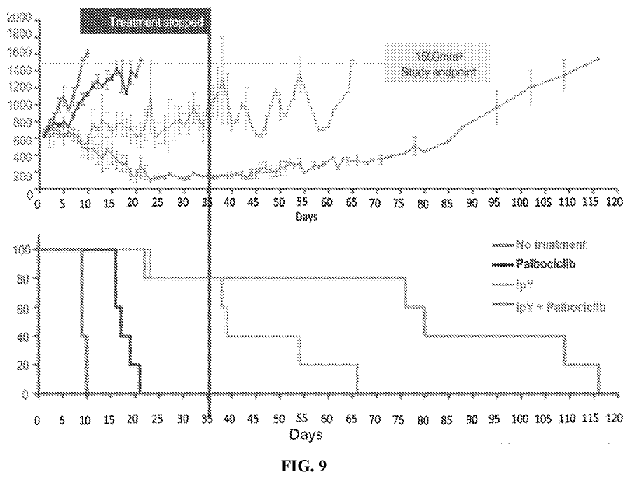

ALT overexpression in breast cancer cells blocks Y88 phosphorylation, inhibiting CDK4, and also preventing p27 degradation, inhibiting CDK2. This arrests cells in G1, induces necroptosis and prevents them from reentering cycle even when ALT was removed, creating a more durable arrest (FIGS. 7A-7C).

Although the invention has been described with reference to the above examples, it will be understood that modifications and variations are encompassed within the spirit and scope of the invention. Accordingly, the invention is limited only by the following claims.

1. A method of treating pancreatic cancer in a subject in need thereof comprising administering to the subject a therapeutically effective amount of a pharmaceutical composition comprising an isolated peptide having the amino acid sequence of SEQ ID NO:6, thereby treating pancreatic cancer in the subject.

2. The method of claim 1, wherein the peptide inhibits cancer cell proliferation and/or decreases cancer cell viability; inhibits tumor growth; increases cancer cell death; increases tumor necrosis; increases cancer cells reactive oxygen species (ROS) level; peptide inhibits phosphorylation of p27; and/or inhibits CDK2 and CDK4.

3-6. (canceled)

7. The method of claim 1, wherein the pancreatic cancer is pancreatic ductal adenocarcinoma (PDAC).

8. The method of claim 7, wherein the PDAC is cyclin-dependent kinase 4/6 (CDK4/6) inhibitor resistant.

9. The method of claim 1, wherein the pharmaceutical composition further comprises a delivery vehicle.

10. The method of claim 9, wherein the delivery vehicle is selected from the group consisting of a nanoparticle, a liposome, a dendrimer, a micelle, a nanoemulsion, a nanosuspension, a niosome, a nanocapsule, a magnetic nanoparticle, a lipoprotein-based carrier, and a lipoplex nanoparticle.

11. The method of claim 10, wherein the lipoplex nanoparticle comprises 1,2-di-O-octdecenyl-3-trimethyl ammonium propane (DOTMA), cholesterol, DOPE, TPGS, or a combination thereof.

12. The method of claim 11, wherein the lipoplex nanoparticle comprises DOTMA and cholesterol, or DOTMA, cholesterol and TPGS.

13-15. (canceled)

16. The method of claim 1, further comprising administering to the subject an anti-cancer treatment.

17. The method of claim 16, wherein the anti-cancer treatment is selected from the group consisting of chemotherapy, radiation treatment, immunotherapy, resection of a tumor, and any combination thereof.

18. The method of claim 16, wherein the anti-cancer treatment is administered prior to, simultaneously with, or after administration of the pharmaceutical composition.

19. The method of claim 17, wherein the anti-cancer treatment is an anti-cancer agent selected from the group consisting of palbociclib, ribociclib, abemaciclib, osirmetinib, gefitinib, lapatinib, pantitumumab, vandetanib, necitumumab, vemurafenib, sorafenib tosylate, PLX-4720, dabrafenib, paclitaxel, cisplatin, docetaxol, carboplatin, vincristine, vinblastine, methotrexate, cyclophosphamide, CPT-11, 5-fluorouracil, gemcitabine, estramustine, carmustine, adriamycin, etoposide, arsenic trioxide, irinotecan, vemurafenib, erlotinib, cetuximab, letrozole, fulvestrant, and epolhilone derivatives.

20. A method of treating pancreatic cancer in a subject in need thereof comprising administering to the subject a therapeutically effective amount of a pharmaceutical composition comprising a cyclin-dependent kinase 4/6 (CDK4/6) and CDK2-specific inhibitor, thereby treating pancreatic cancer in the subject.

21. The method of claim 20, wherein the CDK4/6 and CDK2-specific inhibitor is an isolated peptide having the amino acid sequence of SEQ ID NO:6.

22. The method of claim 21, wherein the peptide inhibits cancer cell proliferation and/or decreases cancer cell viability; inhibits tumor growth; increases cancer cell death; increases tumor necrosis; increases cancer cells reactive oxygen species (ROS) level; peptide inhibits phosphorylation of p27; and/or inhibits CDK2 and CDK4.

23-28. (canceled)

29. The method of claim 20, wherein the pancreatic cancer is pancreatic ductal adenocarcinoma (PDAC).

30. The method of claim 29, wherein the PDAC is cyclin-dependent kinase 4/6 (CDK4/6) inhibitor resistant.

31. The method of claim 20, wherein the pharmaceutical composition further comprises a delivery vehicle.

32. The method of claim 31, wherein the delivery vehicle is selected from the group consisting of a nanoparticle, a liposome, a dendrimer, a micelle, a nanoemulsion, a nanosuspension, a niosome, a nanocapsule, a magnetic nanoparticle, a lipoprotein-based carrier, and a lipoplex nanoparticle.

33. The method of claim 32, wherein the lipoplex nanoparticle comprises 1,2-di-O-octdecenyl-3-trimethyl ammonium propane (DOTMA), cholesterol, DOPE, TPGS, or a combination thereof.

34. The method of claim 33, wherein the lipoplex nanoparticle comprises DOTMA and cholesterol, or DOTMA, cholesterol and TPGS.

35. (canceled)

36. The method of claim 20, further comprising administering to the subject an anti-cancer treatment.

37. The method of claim 36, wherein the anti-cancer treatment is selected from the group consisting of chemotherapy, radiation treatment, immunotherapy, resection of a tumor, and any combination thereof.

38. The method of claim 36, wherein the anti-cancer treatment is administered prior to, simultaneously with, or after administration of the pharmaceutical composition.

39. The method of claim 37, wherein the anti-cancer treatment is an anti-cancer agent selected from the group consisting of palbociclib, ribociclib, abemaciclib, osirmetinib, gefitinib, lapatinib, pantitumumab, vandetanib, necitumumab, vemurafenib, sorafenib tosylate, PLX-4720, dabrafenib, paclitaxel, cisplatin, docetaxol, carboplatin, vincristine, vinblastine, methotrexate, cyclophosphamide, CPT-11, 5-fluorouracil, gemcitabine, estramustine, carmustine, adriamycin, etoposide, arsenic trioxide, irinotecan, vemurafenib, erlotinib, cetuximab, letrozole, fulvestrant, and epolhilone derivatives.
